(19) United States
(12) Patent Application Publication
(10) Pub. No.: US 2017/0209048 A1
(43) Pub. Date: Jul. 27, 2017

(54) REMOTE VITAL SIGN DETECTION DEVICE AND SYSTEM FOR NON-IMPLANTABLE MEDICAL DEVICES AND RELATED METHODS

(71) Applicant: Poiesis Medical, LLC, Jupiter, FL (US)

(72) Inventor: Gregory D. Wiita, Jupiter, FL (US)

(21) Appl. No.: 15/410,717

(22) Filed: Jan. 19, 2017

Related U.S. Application Data
(60) Provisional application No. 62/281,483, filed on Jan. 21, 2016.

(51) Int. Cl. A61B 5/00; A61B 5/20; A61B 5/024; A61B 5/08; A61B 5/01; A61B 5/0215

(52) U.S. Cl. CPC A61B 5/0008; A61B 5/01; A61B 5/6852; A61B 5/0215; A61B 5/024; A61B 5/0816; A61B 5/0084; A61B 5/202

(57) ABSTRACT

A remote vital sign detection device has a sensor, an integrated circuit, and antenna. The integrated circuit is in communication with the sensor and the antenna. The sensor detects and generates an electronic response from a desired vital sign such as body temperature, pulse rate, respiration rate, and blood pressure and sends a signal corresponding to that vital sign to the integrated circuit. The integrated circuit converts the vital sign signal into vital sign data and transmits the vital sign data off-board via the antenna. The vital sign data is then transmitted to an external reader. The external reader receives the vital sign data and processes it for display or read-out by medical staff. An option intermediate receiver may relay vital sign data from the vital sign detection device to the external reader. The vital sign detection device is placed in a non-implantable medical device.

### CROSS REFERENCE TO RELATED APPLICATIONS

This application claims the benefit of U.S. Provisional Application 62/281,483, dated Jan. 21, 2016.

### BRIEF DESCRIPTION OF THE DRAWINGS

FIG. 1 is a schematic of one embodiment of the system of the present invention.

FIG. 2 is a top plan view of one embodiment of the catheter and vital sign detection device of the present invention.

### DETAILED DESCRIPTION

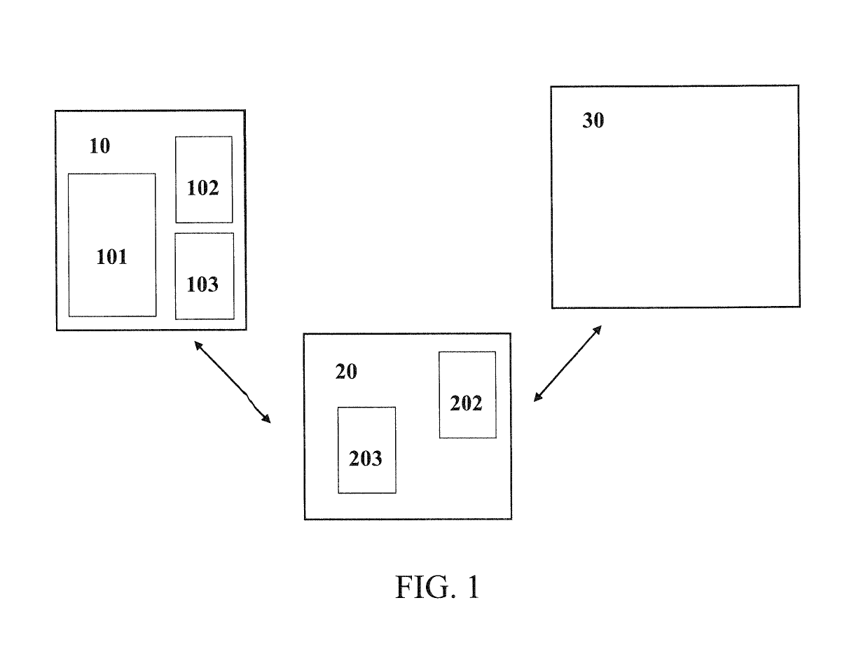

With reference to FIG. 1 shown is a schematic of one embodiment of the system of the present invention. Provided is a vital sign detection device 10, an optional intermediate transmitter 20, and an external reader 30. The vital sign detection device 10 comprises a sensor 101, an integrated circuit 102, and antenna 103. The integrated circuit 102 may be configured as a microchip, microcontroller, or chip as is generally known in the art that includes logic, memory, and instructions to carry out various functions. The integrated circuit 102 is in communication with the sensor 101 and the antenna 103. The sensor 101 is configured to detect and generate an electronic response from a desired vital sign including, but not limited to, body temperature, pulse rate, respiration rate, and blood pressure and send a signal corresponding to that vital sign to the integrated circuit 102. The integrated circuit 102 then can convert the vital sign signal into computer-readable vital sign data and transmit the vital sign data off-board by way of antenna 103. In some embodiments, the computer-readable vital sign data is transmitted first to the intermediate transmitter 20 and then to an external reader 30. The external reader 30 is capable of and configured to receive the computer-readable vital sign data and process it for display or read-out by medical staff.

In some embodiments, the vital sign detection device 10 is configured either in whole or in part as an RFID (radio-frequency identification) device, which may be configured as a tag, chip, module, or the like. In this embodiment, the vital sign detection device 10 uses electromagnetic fields for power and for wirelessly transmitting data. In this embodiment, the device 10 functions as an RFID tag that stores vital sign data obtained from the sensor 101 and then transmits the vital sign data when excited by the intermediate transmitter 20 or the external reader 30. In some embodiments, the vital sign detection device 10 is configured as a passive RFID, active RFID, and/or a battery-assisted passive RFID. The vital sign detection device 10 as configured as RFID may be designed to operate on a plurality of radio communication frequencies, including (1) the low frequency (LF) ranges including 125-134.2 kHz and 140-148.5 kHz; (2) the medium frequency range from 6.765-6.795 MHz and 26.957-27.283 MHz; (3) the high frequency range (known as HF or 13.56 MHz) from 13.553-13.567 MHz; (4) the ultrahigh frequency (UHF) range, including 433 Mhz and from 858-960 MHz; and (5) the super-high frequency (SHF) range, including 2.400-2.483 GHz, 2.446-2.454 GHz, and 5.725-5.875 GHz. The benefit of RFID technology in this application is the relatively small size, low power requirements, wide communication frequency range compatibility, and low cost compared to existing vital sign detection technology. In other embodiments, the vital sign detection device 10 may be configured as a near-field communications-enabled (NFC) device, a Bluetooth-enabled device, a wireless (wifi)-enabled device, or other like wireless communication device including local, remote, and/or networked wireless transmissions capability.

The intermediate transmitter 20 is an optional component but is useful when acting as a communications relay between the vital sign detection device 10 and the external reader 30. Thus, in some embodiments the transmitter 20 comprises an integrated circuit 202 and an antenna 203. The integrated circuit 202 is a microcontroller, microchip or chip that includes logic, memory, and on-board instructions to control the various functions of the intermediate transmitter 20. In some embodiments, particularly where RFID is implemented, the detection range of the vital sign detection device 10 is limited due to its relatively low power nature. Accordingly, in the RFID configuration, the intermediate transmitter 20 functions as a relay and range extender for the vital sign detection device 10 allowing the external reader 30 to activate and read data from the vital sign detection device 10 from a distance otherwise outside of range.

In some embodiments, the intermediate transmitter 20 contains a unique identifier or other authentication data and/or protocol that secures the system components from outside interference and/or unauthorized access. The intermediate transmitter 20 may also function as an RFID reader with respect to the vital sign detection device 10, being capable of generating an electromagnetic excitation signal sufficient to activate the sign detection 10 to retrieve vital sign data therefrom. In some embodiments, the intermediate transmitter 20 can be configured as a near-field communications-enabled (NFC) device, a Bluetooth-enabled device, a wireless (wifi)-enabled device, or other device including local, remote, and/or networked wireless transmissions capable of interfacing with the vital sign detection device 10 through a combination of such transmission and communications protocols. The intermediate transmitter 20 can also be used as an “off-board” primary or supplemental processor for the vital sign detection device 10, which permits the device 10 to have less complicated components and therefore a smaller footprint and lower power requirements. For example, large memory and computing efforts can be handled by the intermediate transmitter 20 while maintaining a communications link with the vital sign detection device 10 in order to efficiently detect, store, and transmit vital sign information. In some embodiments, it follows that the intermediate transmitter 20 can function as a “server” to the “client” vital sign detection device 10.

In some embodiments, external reader 30 is a computing device having a central processing unit, random access memory, storage memory, various input and outputs, and a display. The external reader 30 may be configured as an RFID reader capable of transmitting and electromagnetic excitation signal that activates the vital sign detection device 10 and, optionally, the intermediate transmitter 20 in order to retrieve vital sign data from the vital sign detection device 10 corresponding to one or more vital signs of the patient. The excitation signal may also be strong enough to activate the vital sign detection device 10 if configured as a “passive” RFID meaning it does not have a power source, battery or otherwise.

This functionality allows the vital sign detection device 10 be a small and unobtrusive as possible while also remaining essentially “inert” inside the body. To further enhance the safety and functionality, the vital sign detection device 10 may be encased or encapsulated in silicone, latex, latex coated silicone, and any like and suitable flexible and resilient medical grade material ensuring that no body tissue would be exposed to the device 10

The external reader 30 is capable of interpreting and processing the computer-readable vital sign data received from the sign detection 10 in order to display the vital sign data or otherwise make the vital sign data retrievable and readable by the user. The vital sign data can therefore be displayed directly on the external reader 30 or to an external display or other off-board display device and/or computing device. The external reader 30 also functions to retrieve, interpret, analyze, display and make available vital sign data if Bluetooth, NFC, or local, remote, or networked wireless communications is leveraged along with or instead of RFID. The external reader 30, in some embodiments, can leverage the intermediate transmitter 20 to extend the range of the device 10 and/or distribute processing and transmitting functionality.

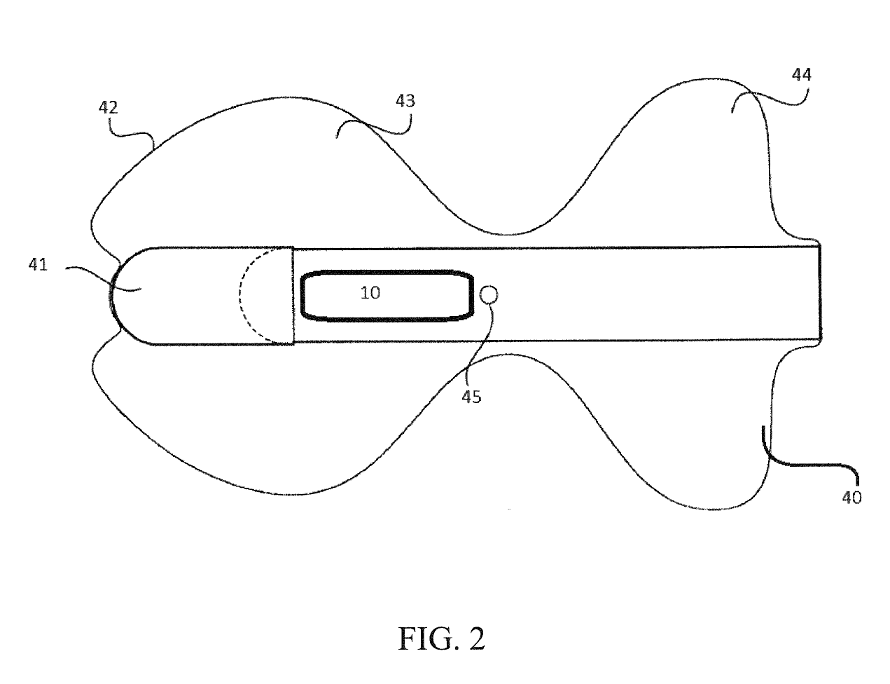

With reference to FIG. 2, shown is a portion of an exemplary non-implantable medical device, catheter 40 that utilizes the vital sign detection device 10 to detect, obtain, and transmit data corresponding to one or more vital signs of a patient. Here, the catheter 40 is configured in accordance with Applicant's invention described in U.S. Pat. No. 8,636,724 and includes a tip end 41 having a cap, a sleeve 42, a first balloon 43 encapsulating the tip end 41, a second balloon 43 to position in the catheter within a body duct, cavity, or vessel of the patient, and a drain port 50. Shown also is vital sign detection device 10 inserted into and disposed adjacent to the tip end 41 of the catheter 40. With the vital sign detection device 10 seated as such, and with the catheter inserted into the bladder or other internal cavity, duct, or vessel of the body, the vital sign detection device 10 is an advantageous position to measure vital signs, such as core body temperature. With sign detection 10 configured as an RFID-capable device, power requirements are non-existent or extremely low, therefore minimizing the impact of the device 10 on the body. Moreover, the device 10 is inserted and protected from the body by way of the catheter 40, providing an ideal way to introduce and retrieve the device 10. It is appreciated that the embodiment shown in FIG. 2 is exemplary in nature as the device 10 can be inserted and disposed in a variety of non-implantable medical devices other than catheters.

In some embodiments, the vital sign detection device 10 may be inserted in the catheter 40, toward the tip end 41 thereof such that a portion of the device 10 is exposed to bodily fluids. This would allow the sensor 101 of the device 10 to perform urinalysis, detect bacteria or colony forming units, and detect bladder inflammation marks, among other procedures and analyses. These features can be available in addition to or instead of the vital sign detection features. In some embodiments, the sensor 101 of the device 10 includes a micro-camera that is exposed to the tip end 41 of the catheter that can be used to assist in placement of the catheter or to otherwise view the position of the catheter and inspect the condition of the bladder or surrounding tissue. Accordingly the vital sign detection device 10 may have several features and characteristics beyond only vital sign detection. As noted above, the vital sign detection device 10 can be encapsulated in silicone to avoid any direct contact with or exposure to tissue or fluids in the body.

With an understanding of the structure of the non-implantable device, the advantages of the intermediate transmitter 20 are apparent in that the range of the sign detection 10 will be limited when disposed within a body. To avoid the need to bring a reader close to or within a cavity of the body to make a reading, in some embodiments, the intermediate transmitter 20 is configured as an adhesive patch that may be disposed non-invasively on the body within readable proximity of the vital sign detection device 10. For example, with the vital sign detection device 10 disposed in a catheter in the bladder of a human, the intermediate transmitter 20 may be adhered to the patient's abdomen such that it is close enough to activate and obtain readings from the device 10. The patch portion of the transmitter 20 can act as an antenna or broadcaster to extend and amplifier the transmission range of the entire system. In other embodiments, the intermediate transmitter 20 can be a portable device or built in to other medical hardware such as a bed, chair, monitor or other like equipment. In other embodiments, the transmitter 20 can be attached to the patient's body at the location where the catheter is externally secured the patient; this is typically an adhesive patch or combination of adhesives that are fashioned around the catheter tube that holds the catheter in place and reducing pulling. In some embodiment, the transmitter 20 can be integrated into an integrated attachment device that combines adhesive features, securement features, and technology features into a single accessory to secure the catheter externally.

In addition to providing vital sign detection and transmission, the present system also may function as an identification and tracking device to determine whether a patient has a catheter installed and the duration of the placement, where it was placed, and the current location of the patient. Realizing that CDC rules mandate that any indwelling catheter be reviewed after 24 hrs as to if it is still medically necessary, this tracking information can be critical to appropriate medical intervention. Considering 15-20% of patients in a hospital will have a catheter during their stay, tracking them is an issue and the present system can provide a robust series of tracking data, either embedded in the vital sign detection device 10, or the intermediate transmission device 20. In some embodiments, the integrated circuit 102 of the device 10 can contain readable/writeable tracking information that can include identification data, start time, stop time, location of placement/procedure, the identity of the medical professional that ordered placement of the catheter, and a data point indicating that the catheter is installed and operating properly. Other relevant tracking, identity, and informational data can be stored in a read-only or re-writable format on the integrated circuit 102 or, in some embodiments, off-board on the intermediate transmitter 20.

While specific embodiments have been described in detail, those with ordinary skill in the art will appreciate that various modifications and alternatives to those details could be developed in light of the overall teachings of the disclosures. Accordingly, the particular arrangements disclosed are meant to be illustrative only and not limiting of the invention, which is to be given the full breadth of the appended claims, and any and all equivalents thereof.

What is claimed is:

1. A remote vital sign detection device, comprising:
a sensor, an integrated circuit, and antenna;
said sensor configured to detect one or more vital signs and send a signal corresponding to said vital signs to said integrated circuit;
wherein said integrated circuit transmits data corresponding to said vital signs by way of said antenna; and
wherein said vital sign detection device is disposed within a non-implantable medical device.

2. The remote vital sign detection device of claim 1, wherein said non-implantable medical device comprises a catheter.

3. The remote vital sign detection device of claim 1, wherein said one or more vital signs comprises body temperature.

4. The remote vital sign detection device of claim 1, wherein said device is configured as an RFID device.

5. The remote vital sign detection device of claim 1, wherein the integrated circuit contains and can transmit identification data, tracking data, duration of placement data, and combinations thereof.

6. A remote vital sign detection system, comprising:
a remote vital sign detection device and an external reader;
said vital sign detection device disposed within a non-implantable medical device;
said vital sign detection device configured to detect one or more vital signs and transmit data corresponding to said one or more vital signs to said external reader.

7. The remote vital sign detection system of claim 6, further comprising an intermediate transmitter configured to relay said data from said vital sign detection device to said external reader.

8. The remote vital sign detection system of claim 6, wherein said data is transmitted by said vital sign detection device when excited by an excitation signal transmitted by said external reader to said vital sign detection device.

9. The remote vital sign detection system of claim 6, wherein said external reader and said vital sign detection device are RFID enabled.

10. The remote vital sign detection device of claim 6, wherein said one or more vital signs comprises body temperature.

11. A method of remotely detecting one or more vital signs, comprising:
providing a remote vital sign detection device comprising a sensor, an integrated circuit, and an antenna;
inserting said remote vital sign into a body cavity, duct, or vessel;
detecting, by way of said sensor, said one or more vital signs;
transmitting a vital sign signal corresponding to said one or more vital signs from said sensor to said integrated circuit;
receiving, on said integrated circuit, an excitation signal; and
in response to said excitation signal, transmitting by said antenna, vital sign data corresponding to said signal.
